(19) United States
(12) Patent Application Publication
(10) Pub. No.: US 2022/0331541 A1
(43) Pub. Date: Oct. 20, 2022

(54) Cannula for the Supply of Oxygen

(71) Applicant: K & J Medical Devices, LLS, Chicago, IL (US)

(72) Inventor: Kate Fontaine, Hobe Sound, FL (US)

(73) Assignee: K&J Medical Devices, LLC, Chicago, IL (US)

(21) Appl. No.: 17/723,250

(22) Filed: Apr. 18, 2022

Related U.S. Application Data
(60) Provisional application No. 63/176,956, filed on Apr. 20, 2021.

(51) Int. Cl. A61M 16/06; A61M 16/08

(52) U.S. Cl. CPC A61M 16/0672; A61M 16/0683; A61M 16/0816

(57) ABSTRACT

The present invention relates generally to cannula that interconnects an oxygen supply to a pair of hoses and two outlets or nasal tips, each tip having a polished opening for delivering oxygen into the nasal passage; the hose member from the supply received by the cannula via a connector to branch two oxygen carrying tubes, threading through a clasp encircling the hoses beneath the chin to stabilize the cannula and position them up or down; the hoses branching to opposing sides of the chin, behind each ear, meeting at a second clasp at the midpoint of the forehead above the bridge of a nose, wherein each tube courses along opposite sides of the nose downwardly into a bend element (i.e., a moldable and/or malleable turn) terminating into an outlet at the Ala's edge coming to rest inside respective nasal passage.

### CLAIM OF PRIORITY

This application claims, pursuant to 35 USC 119, priority to, and the benefit of the earlier filing date of, that patent application filed on Apr. 20, 2021 and afforded Ser. No. 63/176,956, the contents of which are incorporated by reference, herein.

### FIELD OF INVENTION

The present invention relates to a cannula apparatus for nasal insertion for supplying respiratory oxygen to a patient.

### BACKGROUND

Oxygen is prescribed for individuals who are unable to provide for their own requirements for various physically related reasons. For example, supply devices may be an essential diagnostic/therapeutic component of treatment for chronic obstructive pulmonary disease. In this latter instance the supply is a continuing essential part of life. For some individuals, portable oxygen concentrators allow the use of external sources of oxygen, where the wearer of a cannula con move about comfortably, outside their home.

Despite efforts to use new materials, component design, and positioning of the cannula tubing that connects the oxygen supply, current systems have multiple drawbacks that injure through their contact with the dermis over which the tubing travels and the nozzle through which the oxygen is delivered into the nostrils. One effect products pressure ulcers that can cause morbidity in user. These pressure ulcers appear in the shape of the device contacting a patient's soft tissue (See, Black, J. M., Kalowes, P., Medical device-related pressure ulcers, Chronic Wound Care Management and Research 2016: 3, Dovepress, at https://www.doverpress.com/medical-device-related-pressure-ulcers-peer-reviewed-fulltext-article-CWCMR#ref2). According to Black and Kalowes, “The priority placed on the airway often leads to tight, or too loose securements, leading to ulcers by rubbing of the prongs in the nares, for the securement device (e.g., lip ulcers from tape to secure the ET tube), a tight device to maintain a seal (e.g., facial ulcers from non-invasive positive pressure ventilation (“NIPPV”), or tightening the tubing to prevent dislodgement (e.g., tightening up the nasal cannula, leading to ulcers on the ears or face.”

The challenges in producing a NIPPV requires proper material selection, maintaining minimal forces necessary for mechanical stability, and selection of the route over which the tubes are placed and patient comfort. Today, a few different fixed shapes and sizes of standard cannula exist, none of which optimize all the foregoing, but rather maximize the market share via accommodation to the largest variety of users, who need oxygen therapy. As a result, most of the wearers of cannulas are poorly served, making its use uncomfortable, unstable where it moves about the face and nostrils, and does not reliably deliver the prescribed amount of oxygen.

A consequence of a poor fitting cannula affects a significant ambulatory population causing chronic discomfort, sores in the inner side walls of the nose, medial crural footplate, injury to columella, as well as creating lesions above and behind the ears. For example, following a night's sleep, a cannula often leaves indentations of each side of the cheek. Depending on the elasticity of the soft tissue, indentations may take hours to disappear. As ambulatory oxygen therapy users move about during the day, the cannula tubes beneath the chin often kink, reducing the amount of oxygen delivered, creating medical risk and additional discomfort. A constant activity surrounds removing a four-to-seven foot hose from under one's garments, which involves detaching it as a conventional point and untwisting the hose to fix the kink.

Viewing the prior art one concludes that none achieves the objectives discussed, herein, more fully than the invention set forth in the Summary, below. Until the current disclosure, the search for a more comfortable and better functioning product has been frustrating to the user. As mentioned, the prior art has sought to fit the device to a largest variety of users without factoring the inherent features of head, face and particularly nose anatomy, and size.

U.S. Pat. No. 7,156,097 (′097) shows an oxygen delivery system that includes a L shaped piece extending from the oxygen user's forehead down the nose and into a nares, as shown in FIG. 2C. As disclosed in the disclosed invention herein, the oxygen delivery nozzle is not L-shaped. As shown in the first portion of the first claim in ′097, the L-shaped piece is connected at the proximal end to an oxygen source and at the distal end to a nosepiece having ports for intranasal oxygen delivery. In claim 2, the reference gives the option for the L-shaped piece and the nosepiece to be a single component. This reference shows the oxygen tubing with which the L-shaped piece is in fluid communication extends past the forehead, over the oxygen user's head, to a machine in FIG. 6 (not shown). The route of the tubing over the head results in a fit that frequently moves and has to be adjusted.

US Pat. Appl. 2008/0257343 as shown in FIG. 7, is directed to oxygen therapy glasses where the cannula assembly is integrated into the frame of the glasses. Many oxygen users do not need glasses. It also shows the cannula ending immediately below the rim of the glasses where oxygen is supplied to the nares via a j-shaped tube (reference 54 of FIG. 2). This system does not have a Cannu system extending from the oxygen user's forehead to their ears. Also, the mechanism that holds the tubing in place is the portion of the glasses that rests on the oxygen user's nose and thus not mechanically secured and adding pressure to the nose bridge.

As a general matter US Pat. 2, 168, 705 (′705) shows nasal prongs extending from the forehead to the nares visa individual tubes 15. FIG. 2 also shows a bulbous end (16) to the oxygen delivery tube (15) with the oxygen egress going directly out the distal end (see left nasal cannula). The ′705 reference shows the nasal tubes 15 converging in element 13. From there, the tubing goes over the oxygen user's head, which again causes problems with the cannula remaining in place.

US Pat. 4, 559, 941 incorporates, in part, eyeglasses, where the oxygen is delivered via a cannula that extends off the nose piece, not internal to the frame of the glasses.

Similarly, US Pat. 7,942, 150 incorporates, in part, eyeglasses, where the oxygen is delivered via a cannula that extends off the nose piece, not internal to the frame of the glasses.

US PAT. Appl. 2005/0121037 shows an oxygen delivery system wherein the distal portion that enters the oxygen user's nose is a distinct piece that can enter either naris.

U.S. Pat. No. 8,225,796 shows nasal cannula 26 that go into either naris and is distinct from the remainder of the oxygen delivery tubing 14. The rest of this oxygen delivery system seems largely identical to conventional systems, with the tubing traversing the oxygen user's cheek and looping over the ear.

US PAT. Appl. 2005/0092329 shows individual tubes going into each naris in the claims.

U.S. Pat. No. 6,669,712 shows a gas delivery system where delivery tubes are mounted roughly on the forehead. The tubes extend to the oxygen system over the oxygen user's head.

U.S. Pat. No. 7,559,327 shows a variety of distal end designs that are inserted into the nostril.

As the foregoing indicates, many references pertain to oxygen-delivery systems, but none of the cited references show a nonrigid system where the oxygen cannulas extend downward from above an oxygen user's nose without affixing to the patient at any point on the dermis or soft tissue. The cited prior art also does not show retracting a connecting ring from around the site where the j-prong lines joined with the oxygen cannula.

Needless to say, a poorly fitting cannula reduces the amount of oxygen and the quality of the oxygen therapy. Significantly it leads to ulceration and in some cases a shortened lifespan. What is needed is a device that corrects the insufficiencies existing with the prior art.

### SUMMARY OF THE INVENTION

The present invention includes a cannula that connects an oxygen supply to a pair of hoses (or tubing elements) and two outlets or nasal tips, each tip, smoothly shaped, e.g., 0-Tip, teardrop, egg-shaped, tulip, having a polished burnished opening for delivery of oxygen into the nasal passage; the hose member from the supply is received by the two cannula hoses via a connector, which converge in a first channel encircling the hoses to stabilized the hoses allowing movement of each hose up or down proximally at the middle of the chest, (where it connects to the Oxygen supply); the channel including one of a gird, chinch, clasp or bolo tie; the hoses branching to opposing sides of the chinch, behind each ear, towards the forehead, meeting at a second channel proximally at the midpoint of the forehead above the bridge of the nose, where each tube rests on each side of the nose downward into a bend (e.g., U-shaped turn) element terminating into an outlet at the Alar's edge coming to rest inside a respective nasal passage. The ala is the fleshy, curved part of the edge of the nares. Ala flaring describes an ala that has an excess amount of curvature and bows out significantly before it joins the cheek.

In one embodiment of the invention the cannula structure includes a hose or tubing element that connects a supply of oxygen to a pair of hoses and two complementary nasal tips, each tip having an opening for porting oxygen into the nasal passage. The hose from the oxygen supply is received by the cannula via an interface that connects two tubes to the oxygen supply, which tubes thereafter thread through a first clasp to stabilize the cannula in the torso area. The clasp fits around the two hoses to allow the hoses an up or down movement to accommodate user's needs, coming to rest, in a user optional position, beneath the chin. From under the chin the hoses divide coursing to each side of the chin, upward behind each ear, toward the middle of the forehead, where they meet between the eyebrows, joined by a second clasp above the ridge of a nose, but laying alongside of the nose to independently route downward into a bend element (e.g., U-shaped, J-shaped turn) to a terminus that includes the lower end of the tip, which sits at the ala's edge, that is at the edge of the nostril, while the upper tip rests inside of the nostrils.

In one embodiment, the shape of the tip resembles a Q-tip, an egg, a tulip or a teardrop, each approximating an irregular ellipsoid, in either case the opening is formed by a through cut that sections-off the upper portion of the tip. Importantly the tip contains no edges but a generous radius to ensure that the soft tissue within the nasal passage cannot be abraded, chafed or cut.

in another embodiment the inventive cannula facilitates changing the upper part of cannula at the junction via a means at which the cannula attaches to the oxygen supply, allowing for a simplified untwisting of the hoses beneath the chin.

In another embodiment of the present invention includes a method of interconnecting an oxygen supply to a pair of hoses and two outlets or nasal tips, receiving the supply by the two cannula hoses via a connector to the oxygen supply, converging the hoses via a first channel, encircling the hoses to stabilize the hoses, allowing movement of each hose up or down proximally at the middle of the chest, below the chin; branching the hoses to opposing sides of the chin, coursing the each hose behind and over each respective ear, the hoses meeting at a second channel proximally to the midpoint of the forehead above the bridge of the nose, coursing each tube to opposite sides of the nose downward into a bend element (e.g., U-shaped turn), terminating the tips into a respective nasal opening.

In another embodiment the present invention includes a method of wearing a cannula by interconnecting an oxygen supply to a pair of hoses and two outlets or nasal tips, receiving the supply by the two cannula hoses via a connector to the oxygen supply, converging the hoses via a first channel, encircling the hoses to stabilize the hoses, allowing movement of each hose up or down proximally at the middle of the chest, below the chin; branching the hoses to opposing sides of the chin, coursing each hose behind and over each respective ear, the hoses meeting at a second channel proximally at the midpoint of the forehead above the bridge of the nose, coursing each tube to opposite sides of the nose downward into a bend element (e.g. U-shaped, J-shaped turn), terminating the tips into a respective nasal opening.

In one aspect of the invention, the bend element may be integral to a proximal end of a corresponding one of the hoses. In another aspect of the invention, the bend element may be removably attachable to a corresponding one of the hoses or tubing elements.

In one aspect of the invention, the bend element may be of a rigid material that may be molded into a desired form (e.g., U-shaped, J-shaped, etc.). In another aspect of the invention the bend element 24 may be composed of a malleable or flexible material that may be formed into a desired form (e.g., U-shaped, J-shaped, etc.). In another aspect of the invention, the bend element may comprise a bendable region (e.g., an accordion) that allows for the shaping of the bend element into a desired form (e.g., U-shaped, J-shaped, etc.).

In one aspect of the invention, the pair of hoses (or tubing elements) may be constructed of a rigid material wherein the tubing elements are shaped to extend substantially horizontally with respect to a patient's or user's forehead and then substantially vertically to enable the hoses to be positioned anteriorly or posteriorly with respect to the torso of the patient. In one aspect of the invention, the direction of the tubing elements may be altered so that the tubing elements 6a, 6b form an acute angle with respect to the substantially horizontally positioned of tubing elements 6a, 6b. In this manner, tubing elements 6a, 6b may be directed anterior to the user's torse. In another aspect of the invention, the direction of tubing elements 6a, 6b, may be altered so that tubing elements 6a, 6b form an obtuse angle with respect to the substantially horizontally positioned tubing elements 6a, 6b. In this matter, tubing elements 6a, 6b may be directed posterior to the user's torso.

In one aspect of the invention, the pair of hoses may be constructed of a semi-rigid material that may be molded into a shape that causes the tubing elements to extend substantially horizontally with respect to a patient and then substantially vertically to enable the hoses to be positioned anteriorly or posteriorly with respect to the torso of the patient.

In one aspect of the invention the pair of hoses 6a, 6b may be constructed of a conventional flexible material, wherein the clasps 5, 10 may be used to control a shape of the cannula formed by the conventional flexible tubing material.

### BRIEF DESCRIPTION OF THE DRAWINGS

FIG. 1 is a perspective view of a first exemplary embodiment of a cannula in accordance with the principles of the present invention.

FIG. 2 is a top view of a cannula in accordance with an embodiment of the present invention.

FIG. 3 is bottom view of a cannula in accordance with an embodiment of the present invention.

FIG. 4 is a perspective view of the nasal tips of the cannula in accordance with an embodiment of the present invention.

FIG. 5 is a perspective view of a first exemplary embodiment of a clasp leading into the nasal tips of the cannula in accordance with principles of the present invention.

FIG. 6 is a perspective view of a cannula worn by a subject, user or patient in accordance with an embodiment of the present invention.

FIG. 7 is a perspective view of a second exemplary embodiment of a cannula in accordance with the principles of the invention.

FIG. 8 is a front view of the clasp element controlling the position of the tubing elements shown in FIG. 7.

### DETAILED DESCRIPTION OF THE INVENTION

The following detailed description includes the best mode of carrying out the invention and is made for the purpose of illustrating the general principles of the invention and should not be taken in a limiting sense. The scope of the invention is determined by reference to the claims. Each or function is assigned, even if structurally identical to another part, a unique reference number wherever that part is shown in the drawing figures.

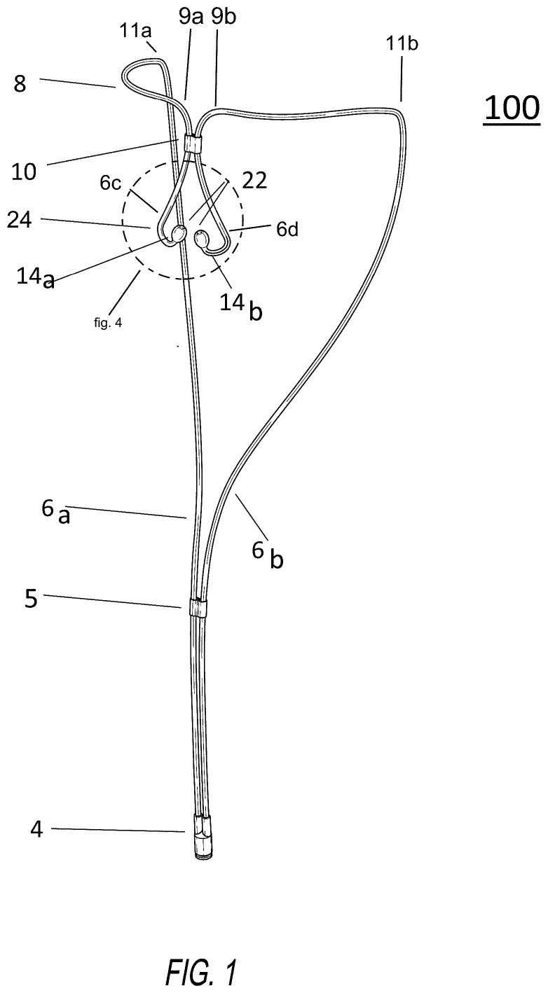

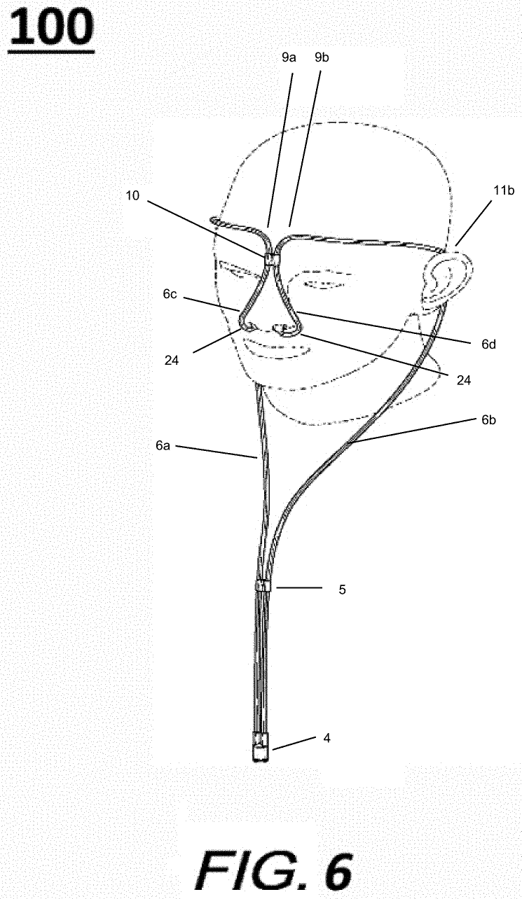

Turning to FIG. 1 and to FIG. 6, the cannula 100 interconnects an oxygen supply (not shown) at connector 4 to a pair of hoses, 6a, 6b, which terminate at two outlets or nasal tips, 14a, 14b, each tip egg-shaped, tulip-shaped or teardrop-shaped having a polished burnished opening 22 for delivering oxygen into the nasal passage; the hose member from the oxygen supply (not shown) is received by the two canula hoses 6a, 6b optimally via a swivel connector (not shown), which is insertable (or connectable) into connector 4. The hoses 6a, 6b extend from connector 4 and pass through a first channel or clasp 5, encircling the hoses 6a, 6b to stabilize them, yet allowing movement of each hose 6a, 6b up or down within the clasp 5, which rests proximally at the middle of the chest; the hoses 6a, 6b, branching to opposing sides of the chin (see FIG. 6), behind and over each ear, meeting at a second channel or clasp 10 proximally at a midpoint of the forehead above the bridge of the nose, where each tube or hose 6a, 6b (referred to as 6c, 6d, respectively) courses on opposite sides of the nose downward into a bend element (e.g., U-shaped turn) 24 terminating into an outlet at the ala's edge, where tips 14a, 14b come to rest inside a respective nasal passage, to allow oxygen to enter via ports 22. Clasps 5, 10 referred to above may include one of a simple ring, a gird, cinch, clasp or bolo tie.

The cannula 100 has a structure with a connector 4 opening (facing down) to receive a connector from an oxygen supply. The connection to connector 4 may be one of a threaded connection, a snap-fit connection, a bayonet connection, etc., The hoses 6a, 6b, upon leaving the connector 4, rest between middle of the chest or under the chin, depending on the size of the user. As illustrated in FIG. 3, from the opposite end of the connector 4, (facing upward) the two hoses 6a and 6b, separate into two narrow tubes, about 2.5 feet long. In one non-liming embodiment the two hoses 6a, 6b passthrough clasp 5 (FIG. 1), which is approximately one-half (½) inch long. The clasp 5 may rest on the torso (anterior or posterior) or may be positioned under the user's chin to stabilize the cannula 100. Thus, clasp 5 may be positionable (i.e., moved up or down) with respect to tube elements (i.e., hoses) 6a, 6b to accommodate a user's needs.

Hoses 6a, 6b further divide and travel to each side of the user's chin, up behind and over each ear, and wherein a first bend 11a, 11b, respectively, may be formed to alter a direction hoses 6a, 6b substantially horizontally towards the middle of the forehead, and meet between the eyebrows at clasp 10 as shown in FIG. 6. In addition, hoses 6a, 6b may include a second bend 9a, 9b, respectively that is formed to alter the direction of hoses 6a, 6b to enable hoses 6a, 6b to passthrough clip 10 and process along the user's nose.

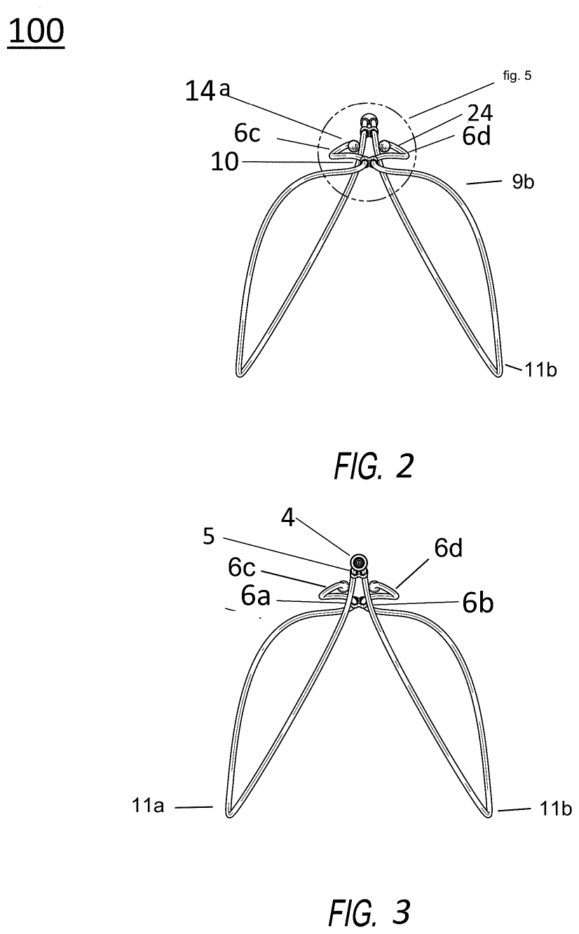

FIG. 2 illustrates a top view of cannula 100 (i.e., a view facing downward from the forehead FIG. 6), above the bridge of the user's nose. In one aspect of the invention, hoses 6a, 6b may be constructed of a rigid (or semi-rigid) material such that neither hose 6a, 6b contacts a corresponding one of the user's ear. In another aspect of the invention, hoses 6a, 6b may be conventional flexible tubing elements that may be positioned behind a user's ear.

The clasp 10 in one non-limiting is structurally a plastic ring, which holds each hose loosely above or at the bridge of the nose. This region is referred to as the glabella, the area of skin between the eyebrows and above the nose. The term also refers to the underlying bone that is slightly depressed and joins the two brow ridges. The hoses 6c, 6d (shown in FIG. 6, for example,) are arranged to be positionable alongside of the nose and at the base of the nose and formed in a bent element (e.g., U-shaped configuration) 24 as they enter at the ala's edge, i.e., at the edge of the nostril, where tips 14a, 14b come to rest inside a respective nasal passage to deliver oxygen to the nostrils.

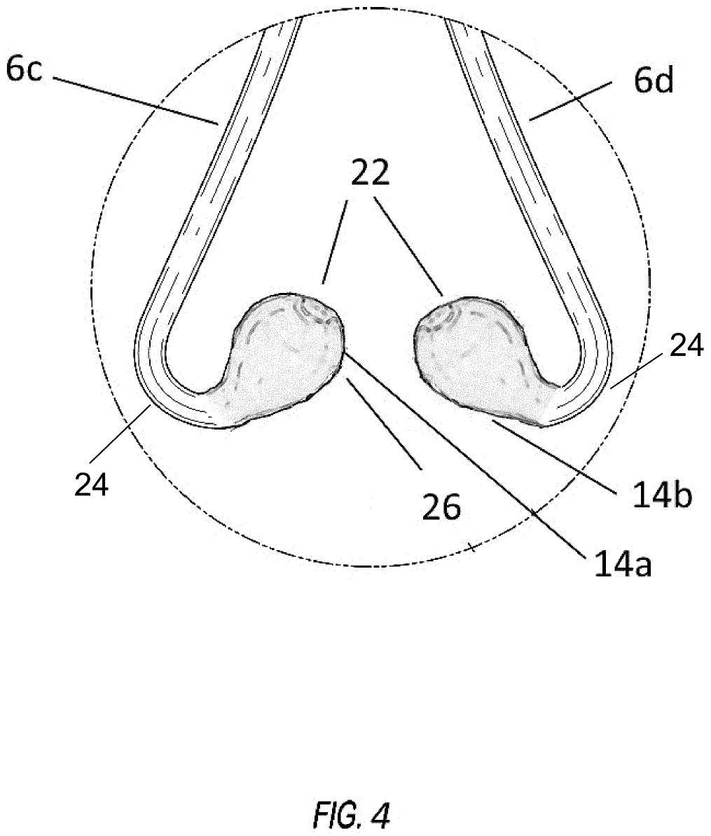

For some individuals, the alar edges are thicker and/or of varying hardness as a consequence of the connective tissue in that area. However, the soft, pliable, material of the cannula tip 14a,14b conforms to the wearer's physiology inside the nostril, where abrasions, as is known in the art, are more likely to occur utilizing conventional tubing systems. The bend element (e.g., U-shape) area 24, shown in detail in FIG. 4, hugs the ala edge. Because it is soft, the bend element area 24 adapts itself to the variety of alar widths or thicknesses. Once the tips 14a, 14b are fit or set into the nostril, they are fixed in place because the plastic ring 10 above the glabella (see FIG. 6) fits snuggly around the two hoses, where they meet above the glabella, dividing along each side of the nose to hug the alar ridge as a result of its U-shape configuration 24. In this way the length between the glabella and the bend element area 24 is maintained. The tips 14a, 14b are bent, such that the cannula cannot be moved up toward the forehead. However, the bend element area 24 can hug any area on the ala edge, from the tip of the nose to the base on the nose, depending on the comfort of the user. Once that is established or set the bend element area 24 is determined and the clasp or ring 10 at the glabella is also held in position.

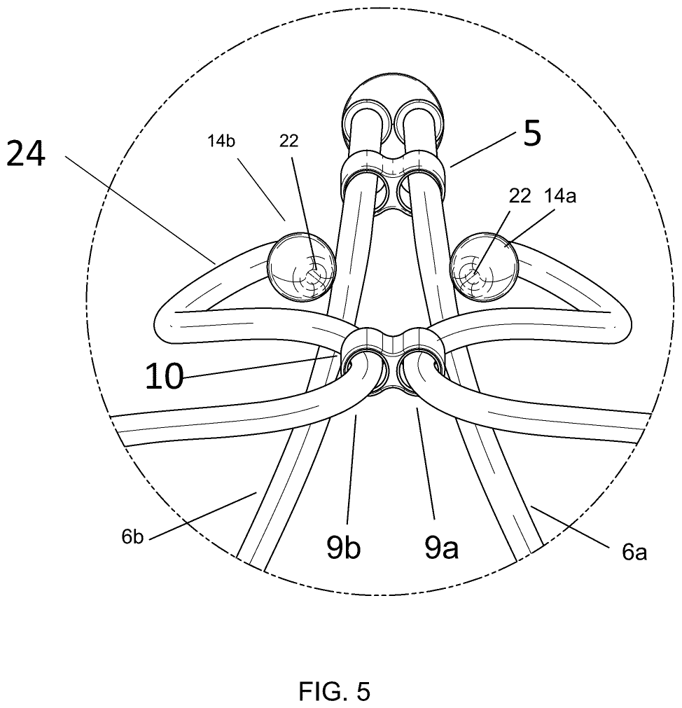

FIG. 5 shows, in detail, the first exemplary (and non-limiting) embodiment of the U-shaped 24 area as well as the hose 6a, 6b serving as one continuous hose member. This continuous feature of the hose member eliminates all the sharp edges to ensure that the area around the base of the nose and at the ala's edge of the nostril are free from any potential for an abrasive injury. This is carried through to the integration of the tip 14a, 14b themselves, where the specially shaped soft tip insures no sharp corners or edges. Each tip 14a, 14b may be burnished toward the inside center of the opening 22 of the tip structure.

Further shown is a first exemplary embodiment of clasp 10 wherein clasp 10 comprises two cylindrical elements joined together that allow hoses (i.e., tubing element) 6a, 6b to pass through and be held in place. In this illustrated embodiment, the illustrated clasp 10 is referred to as “I” shaped clasp, as hose elements 6a, 6b may be slide upward or downward with respect to the clasp 10.

In FIG. 4, the nasal tips 14a, 14b are to be manufactured from a soft, pliable material to adapt to its environment inside the nostril. In this illustrated embodiment, tips14a, 14b have and ellipsoidal shape 26, which resembles that of an egg-shape, a tulip-shape or a teardrop-shape, to minimize pressure point contact with the walls of the nostrils, thus, further reducing or preventing surface irritation or abrasion type injuries. Alternatively, tips 14a, 14b may be of a rounded or spherical shape to minimize pressure point contact with the walls of the nostrils.

Turning to FIG. 6, in this illustrated embodiment, the cannula 100 may be constructed in several sizes of tips; large, medium, and small, for an adult user and smaller sizes for children including pediatrics, Other factors may influence the length of tubing, 6a, 6b, for example, a.) where the ring (10, 70) sits on the forehead; b.) the comfort at the forehead; c.) the forehead's shape; d.) the glabella position; e.) the brow ridge position; f.) shape and the protrusion of the brow; g.) any forehead grooves; h.) wrinkles; and i.) the condition of the user's skin.

Also illustrated in FIG. 6 is a non-limiting embodiment of the present invention of a method of wearing a cannula 100 by interconnecting an oxygen supply to a pair of hoses 6a, 6b and two outlets or nasal tips 14a, 14b, receiving the supply by the two cannula hoses via a connector 4 to the oxygen supply (not shown), converging the hoses 6a, 6b via a first channel in the torso area, encircling the hoses with a clasp 5 to stabilize hoses,6a, 6b allowing movement of clasp 5 up or down proximally at the middle of the chest, below the chin; branching the hoses 6a, 6b to opposing sides of the chin, coursing each hose behind and over each respective ear, without making ear contact, the hoses meeting at a second clasp 10, proximate a midpoint of the forehead above the bridge of the nose, coursing each hose 6a, 6b to opposite sides of the nose downward into a U-shaped turn 24, terminating the tips 14a, 14b into a respective nasal opening. [0043] In another exemplary embodiment, the present invention includes a method of interconnecting an oxygen supply to a pair of hose 6a, 6b to two outlets or nasal tips 14a, 14b, receiving the supply by the two cannula hoses 6a, 6b via a connector 4, converging the hose pairs via a first clasp 5, encircling the hoses to stabilize the hoses, allowing movement of each hose up or down proximate to the middle of the chest; branching the hoses to opposing sides of the chin, courses each hose behind and over each respective ear, the pair of hoses 6a, 6b meeting at a second clasp 10, proximate to the midpoint of the forehead above the bridge of the nose, coursing each tube 6a, 6b to opposite sides of the nose downward into a bent shaped element, terminating the tips 14a, 14b into respective nasal openings.

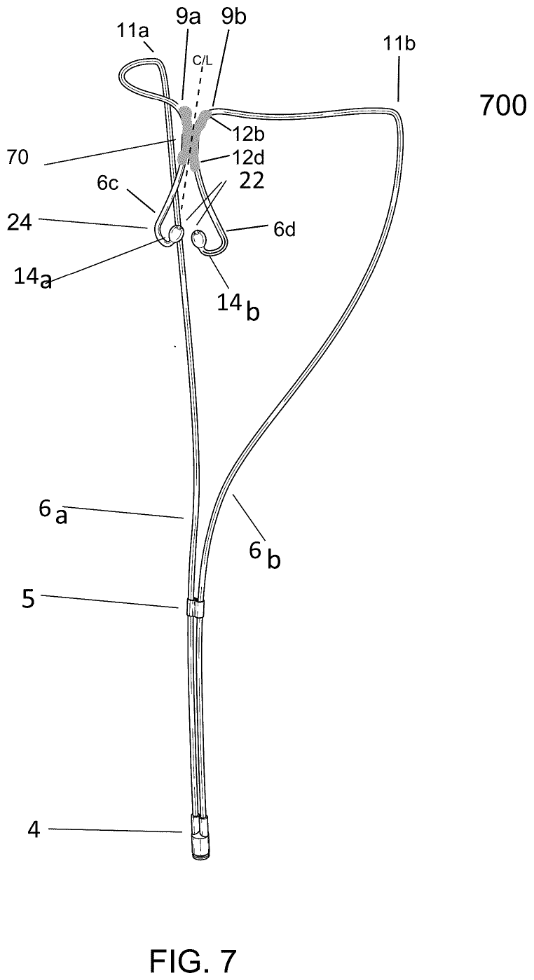

FIG. 7 illustrates a second exemplary embodiment 700 of a cannula in accordance with the principles of the invention.

In this second exemplary embodiment, which is similar to the embodiment shown in FIG. 1, hoses 6a, 6b proceed from connector 4 through first clasp 5 and second clasp 70 to tips 14a, 14b, which are inserted into the nostrils of the user.

In this illustrated embodiment, clasp 70, which is shaped in the form of a bent-tipped “X” allows for the control of the placement of hoses 6a, 6b with respect to a patient's brow and nose. For example, the upper ends of bent-tipped clasp 70 may be shaped to enable hoses 6a, 6b to be positioned substantially horizontal to the user's eyebrows (i.e., horizontal to a patient, perpendicular to the patient's torso) while the lower ends of clasp 70 may be shaped to cause hoses 6a, 6b to extend along the user's nose. In one aspect of the invention, the bent-tipped clasp 70 illustrated in FIG. 7 may be molded (or formed) into the illustrated bent tipped “X” (or “X”) shape. In another aspect of the invention, illustrated clasp 70 may be composed of a malleable material that allows for the manipulation of the illustrated “X” shape, (i.e., separated upper and lower tips have substantially same angle separation with respect to a vertical center line) or allow for the formation of a “Y” shaped clasp (i.e., separated upper tips have a greater angular separation than the lower tips with respect to a vertical center line).

In one aspect of the invention, clasp 70, as shown may be in the form of a bent-tip “X” element, that is formed to support a conventional tubing element (or hoses) 6a, 6b used in current cannula designs. In this illustrated exemplary embodiment, the upper tip elements 12a, 12b, suitable for connecting hoses 6a, 6b are oriented at approximately 15 degrees from a center line (represented by center line C/L) to enable hoses 6a, 6b to be positioned substantially horizontal to the user. Similarly lower tips 12c, 12d are oriented at approximately 15 degrees from the center line of clasp 70 to enable hoses 6a, 6b to be positioned along opposite sides of the user's nose.

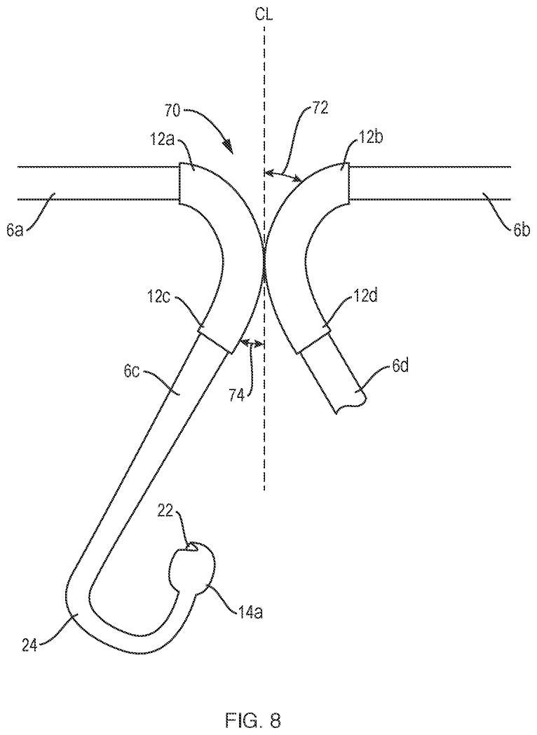

FIG. 8 illustrates a front view of the bent-tipped clasp 70 illustrated in FIG. 7 showing the orientation of the tips 12a, 12b, 12c and 12d with respect to a center line (C/L). In one aspect of the invention, clasp 70 may be formed of a rigid material, wherein the orientation of tips 12a-12d may be fixed with respect to center line C/L. In another aspect of the invention, clasp 70 may be formed of a malleable (or semi-rigid) material that allows the angle that tips 12a-12d expressed with center line C/L to be independently adjusted. For example, tips 12a and 12c may be positioned to retain tubing element 6a, 6d (respectively) substantially perpendicular to center line C/L as shown. Alternatively, the orientation of tips 12a and 12d may be positioned based on the comfort of the user.

Similarly, tips 12c and 12d may be positionable to retain that portion 6c, 6d of tubing element 6a, 6b, respectively to align along the nose of the user.

Thus, although the tips 12a-12d of clasp 70 are shown in FIG. 8 with regard to an orientation of approximately 15 degrees with respect to center line C/L, it would be understood that this is only one configuration and is not intended to be the only orientation of either the upper tips 12a, 12b or the lower tips 12c, 12d.

In summary, the disclosed invention provides a new and innovative cannula design that addresses the deficiencies in the prior art according to the following objects: a.) to deliver a prescribed amount of oxygen; b.) to improve mental health and well-being to a user due to ease of use, comfort, and visual appearance; c.) to improve hygiene through the reduction of nose-touching to adjust the cannula; d.) to adjust the cannula to reliably fit a variety in sizes and widths of noses, e.) to allow greater freedom of movement as the neck and face move, such as turning one's head, eating, speaking, laughing or smiling; f.) to reduce facial indentations; g.) to decrease the occurrence of irritations around the ears, including the reduction in the formation of calluses and the frequency of nose bleeds; h.) and compared to the prior art utilize less material, and thus lessening the environmental impact upon disposal.

While the foregoing invention has been described with reference to the above embodiments, additional modification and changes can be made without departing from the spirit of the invention.

What is claimed is:

1. A oxygen delivery system comprising:
a pair of tubing elements, each of said pair of tubing elements comprising:
a distal end; and
a proximal end,
a first clasp configured to retain said pair of tubing elements, wherein said first clasp is slidably positionable with respect to said pair of tubing elements;
a second clasp configured to retain said pair of tubing elements, wherein each of said pair of tubing elements being slidably positionable with respect to said second clasp;
a bend element substantially adjacent to said proximal end of a corresponding one of each of said pair of tubing elements; and
a bulbous element positioned on said proximal end of each of said bend element.

2. The oxygen delivery system of claim 1, comprising: a connector interface, wherein said distal end of each of said pair of tubing elements is insertable into said connector interface.

3. The oxygen delivery system of claim 1, wherein said bend element is one of: integral to a corresponding one of said pair of tubing elements and removably attachable to a corresponding one of said pair of tubing elements.

4. The oxygen delivery system of claim 1, wherein said bend element is adjustable.

5. The oxygen delivery system of claim 1, wherein said bend element is shaped as one of: a “U” shape and a “J” shape.

6. The oxygen delivery system of claim 1, wherein said bend element is one of: rigid, semi-rigid and flexible.

7. The oxygen delivery system of claim 1, wherein said bulbous element is one of: round, ellipsoidal shaped, egg-shaped, tulip-shaped and teardrop-shaped.

8. The oxygen delivery system of claim 1, wherein said bulbous element is composed of a pliable material.

9. The oxygen delivery system of 1, wherein said second clasp comprises:
a pair of passthrough elements, wherein each of said pair of passthrough elements is one of: straight, curved and a combination thereof.

10. The oxygen delivery system of claim 1, wherein said second clasp is shaped as one of: a “X” shape and a “Y” shape.

11. The oxygen delivery system of claim 1, wherein said second clasp is one of: a ring, a gird, and a bolo tie

12. An oxygen delivery system comprising:
a first tubing element;
a second tubing element, each of said first tubing element and said second tubing element comprising:
a bend element substantially adjacent a first end of a corresponding one of said first tubing element and said second tubing element; and
a bulbous element positioned on said first end of a corresponding one of first tubing element and said second tubing element;
a first clasp joining said first tubing element and said second tubing element; and
a second clasp joining said first tubing element and said second tubing element, wherein said first tubing element and said second tubing element are positionably adjustable within said second clasp.

13. The oxygen delivery system of claim 12, wherein said bulbous element comprises a pliable material and is one of: round, ellipsoidal shaped, egg-shaped, tulip-shaped and teardrop-shaped.

14. The oxygen delivery system of claim 12, wherein said bend element is one of: rigid, semi-rigid and flexible.

15. The oxygen delivery system of claim 12, wherein said second clasp is one of: “I”-shaped, “X”-shaped and “Y” shaped.

16. An oxygen delivery system configured to provide oxygen to a patient, said oxygen delivery system comprising:
a pair of tubing elements, extending along a torso of said patient, wherein each of said pair of tubing elements comprises:
a bend element proximate to a proximal end of a corresponding one of said pair of tubing elements; and
an ellipsoidal shaped bulbous element positioned on said proximal end of a corresponding one of said pair of bend elements, said bulbous element being composed of a pliable material, wherein said bulbous element is configured to engage a nostril of said patient;
a distal end clasp positionable with respect to said pair of tubing element along said torso of said patient;
a proximal end clasp positionable above a nose bridge of said patient, wherein said pair of tubing element extend through said proximal end clasp and along a nose of said patient, wherein said pair of tube element are positionable with respect to said proximal end clasp to cause said bulbous element to engage a corresponding nostril of said patient.

17. The oxygen delivery system of claim 16, wherein said pair of tubing elements is one of: flexible, semi-rigid and rigid.

18. The oxygen delivery system of claim 16, wherein said proximal end clasp is one of: “I”-shaped, “X”-shaped and “Y”-shaped.

19. The oxygen delivery system of claim 16, comprising:
a connector interface element, wherein said distal end of each of said pair of tubing elements is positioned within said connector interface element and said connector interface element is configured to engage an oxygen supply system.

20. The oxygen delivery system of claim 16 wherein said pair of tubing elements comprises:
a second bend element proximal to said proximal end clasp, wherein said second bend element is configured to:
direct said pair to tubing elements substantially horizontal to said patient; and
a third bend element configured to alter a direction of each of said pair of tubing elements, wherein said alteration is one of: acute to and obtuse to said substantially positioned pair of tubing elements.
